(19) United States
(12) Patent Application Publication
(10) Pub. No.: US 2007/0073397 A1
(43) Pub. Date: Mar. 29, 2007

(54) Disc nucleus prosthesis and its method of insertion and revision

(76) Inventor: Laurence M. McKinley, Escondido, CA (US)

Correspondence Address: CHRISTIE, PARKER & HALE, LLP, PO BOX 7068, PASADENA, CA, 91109-7068, US

(21) Appl. No.: 11/228,618

(22) Filed: Sep. 15, 2005

(51) Int. Cl. A61F 2/44

(52) U.S. Cl. 623/17.11

(57) ABSTRACT

A revisable nuclear disc prosthesis and method of insertion and revision are provided. The revisable disc prosthesis includes a reversibly expandable support cage surrounding a containment vessel containing a suitable structural support material, and a hydrodynamic filler material disposed in the disc space between the disc prosthesis support cage and the annulus of the body. Each piece of the disc prosthesis is capable of removal and revision once inserted into the body of the patient.

### FIELD OF THE INVENTION

The current invention is directed to a prosthetic discs; and more particularly to revisable nuclear and total disc prostheses.

### BACKGROUND OF THE INVENTION

Spinal discs are divided into two basic parts: an outer, peripheral portion, known as an annulus fibrosis, and a center, known as the nucleus pulposus or “nucleus.” The nucleus acts to support annulus, which transmits torque between neighboring vertebrae. The annulus is made up of around 13-15 lamellae, which cross each other at an approximate angle of 67° similar to a cross-ply tire. If the annulus is not properly supported in its center, the height of the disc space can collapse and the annulus lamellae can delaminate. The annulus can then tear and its torque transmission can weaken.

The nucleus is composed of a mesh of proteoglycon molecules embedded in collagen fibers. It has a pulpy, watery consistency, which is substantially incompressible. The nucleus supports the annulus by rolling slightly forward and backward between the cartilaginous end plates of neighboring vertebrae with an instantaneous center of rotation that moves around 3-4 mm in flexion and extension. The nucleus therefore provides a polycentric pivot point in the motion of the disc segment, and exerts an external pressure against the inner fibers of the annulus, preventing distortion, delamination, and buckling. Initial attempts to replace the disc nucleus have ended in failure because all of the replacement materials developed thus far have been unable to withstand the repeated forces applied to the material

As a result, doctors have moved away from addressing herniated discs by replacement of just the nucleus of the disc to techniques that require the removal of the entire disc. The disc space is then typically distracted by either insertion of a piece of bone, that then fuses the neighboring vertebrae together, or a prosthetic disc. Both of these techniques are fraught with peril. Interbody fusion, increases the stress on other vertebrae and often leads to premature adjacent level disc degeneration. Likewise, conventional prosthetic discs tend to decrease in height towards their centers after time, causing looseness in the ligamentous connections on the outside of the spine. This looseness allows movement of the prosthetic disc, decreasing the effectiveness of the torque transmission and the fixation of the prosthetic device between the neighboring vertebrae. Other prosthetic discs that have a higher force-per-unit-area, such as a ball-bearing-style disc, can withstand compressive forces well, but tend to push into adjacent endplates, decreasing the height of the intervertebral disc space.

Accordingly, a need exists for an improved prosthetic disc system capable of providing a long-term solution to disc replacements.

### SUMMARY OF THE INVENTION

The current invention is directed to a revisable nuclear disc prosthesis.

In one embodiment, the revisable disc prosthesis includes a reversibly expandable support cage. In one such embodiment, the reversibly expandable support cage comprises a plurality of staves connected at either end to a separate cooperatively threaded end cap such that threading together the two end caps forces the staves to expansively distort outwardly creating and expanded support cage.

In another embodiment, the support cage of the disc prosthesis of the current invention is made of a memory alloy such that during expansion the staves are released into a arbitrary pre-set memory position.

In still another embodiment, the revisable disc prosthesis includes a containment vessel disposed within the support cage for receipt of a support material. In one such embodiment, the containment vessel is filled with a synthetic cancellous bone-void material, such as beta-tricalcium phosphate.

In yet another embodiment, the revisable disc prosthesis includes a hydrodynamic filler material disposed in the disc space between the disc prosthesis support cage and the annulus of the body. In one such embodiment, the hydrodynamic filler material is formed of a synthetic polymer, such as a polyvinyl, pyrolidone, polyvinyl alcohol, poly-2-hydroxylethylmethacralate, or polysiloxane modified styrene-ethylene-butylene block co-polymer.

In still yet another embodiment, the invention is directed to a method of posterolateral insertion of the disc nucleus prosthesis of the current invention.

In still yet another embodiment, the invention is directed to a method of revision of the disc nucleus prosthesis of the current invention. In one such embodiment, the method includes the step of ultrasonically emulsifying the structural filler material disposed within the containment vessel of the disc prosthesis.

### BRIEF DESCRIPTION OF THE DRAWINGS

These and other features and advantages of the present invention will be better understood by reference to the following detailed description when considered in conjunction with the accompanying drawings wherein:

FIG. 1a provides a schematic side cross-sectional view of a disc;

FIG. 1b provides a schematic top cross-sectional view of a disc;

FIG. 2 provides a schematic of a disc prosthesis in accordance with one embodiment of the current invention;

FIG. 3a provides a schematic of a conventional Molly-type expansion bolt in an unexpanded state;

FIG. 3b provides a schematic of a conventional Molly-type expansion bolt in an expanded state;

FIG. 4a provides a schematic of an unexpanded disc prosthesis cage in accordance with one embodiment of the current invention;

FIG. 4b provides a schematic cross-sectional view of an unexpanded disc prosthesis cage in accordance with one embodiment of the current invention;

FIG. 4c provides a schematic of an expanded disc prosthesis cage in accordance with one embodiment of the current invention;

FIG. 5a provides a schematic cross-sectional view of an exemplary disc space;

FIGS. 5b and 5c provide schematic side and top views of a conventional disc prosthesis cage after insertion into the disc space of FIG. 5a;

FIG. 5d provides a schematic top view of a disc prosthesis cage in accordance with the current invention after insertion into the disc space of FIG. 5a;

FIG. 6a and 6b provide schematics an inserted disc prosthesis along with a containment vessel and method of insertion thereof, in accordance with the current invention;

FIGS. 7a and 7d provide schematic views of the backfill of the annulus stabilizing material between the disc prosthesis cage and the annulus in accordance with the current invention;

FIGS. 8a to 8d provide schematic views of an exemplary method of insertion of the disc prosthesis of the current invention into a patient; and

FIGS. 9a to 9d provide schematic views of an exemplary method of revision of the disc prosthesis of the current invention.

### DETAILED DESCRIPTION OF THE INVENTION

The current invention is directed to a disc prosthesis; and more particularly to a controllably expandable nuclear prosthesis capable of post-surgery revision.

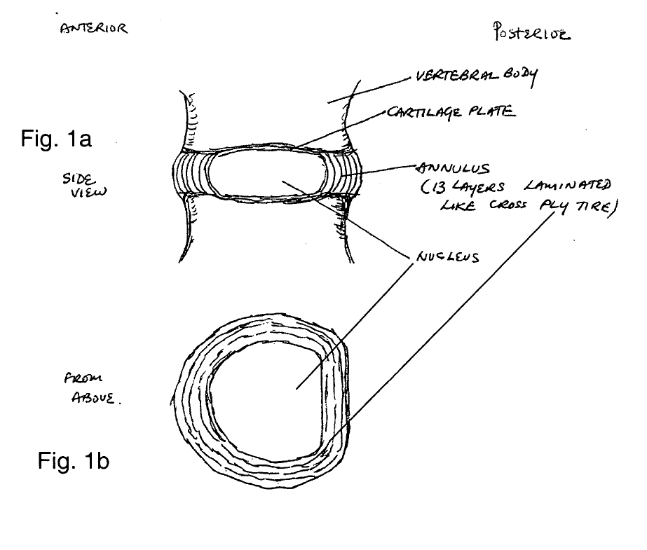

Before discussing the structure and operation of the disc prosthesis of the current invention, it is important to consider the mechanics of the intervertebral disc, and understand its physical limitations. The normal intervertebral disc is a very specialized joint between two vertebral bodies. FIG. 1a provides a cross-sectional diagram of a normal intervertebral disc. The disc is made up of a strong outer ring called the annulus, which has 13 to 15 lamellae, which intersect with each other at an angle of about 67 degrees to produce a cross-ply construct, as shown in a top cross-sectional view in FIG. 1b. This formation and the alignment of annular fibers allows for the most mechanically efficient transference of torque. However, if the annulus is not properly supported from within, the disc height collapses and the annulus lamellae delaminate creating tears, which in turn produces weakness in the torque transmission. Inside the annulus is the nucleus pulposus. The nucleus pulposus is composed of a mesh of proteoglycon molecules embedded in collagen fibers. The nucleus is very hydroscopic; approximately 98% water; and as a fluid medium is incompressible. The nucleus material is also capable of moving about in the intervertebral disc space to provide a polycentric pivot point in the motion of the disc segment. Finally, the nucleus exerts an external pressure against the inner fibers of the annulus, supporting the annulus, thereby preventing distortion, delamination and buckling.

In a standard disc excision surgery, the annulus is weakened by the annulotomy (the aperture formed in the disc to permit entry to obtain the nuclear material). The fibers of the lamellae orientation in the annulus are likewise transected. The nucleus is removed, and the vertical height of the disc is reduced, resulting in the bulging of the lamellae and the potential for delamination, reducing the ability of the annulus to transmit torque from one vertebral segment to another. This compromises the motion segment and leads to post-discectomy segmental instability, a common source of mechanical back pain. Accordingly, the basic requirements of a disc prosthesis are:
1. It should be biocompatible, and reproduce as closely as possible the normal disc geometry, kinematics, and dynamics. In short, the device must maintain the proper intervertebral spacing, keeping the annular fibers under tension. The device should be able to act as a shock absorber, and should not shift significantly or extrude into the spinal canal, causing a syndrome similar to repeat disc herniation.
2. It should provide some type of motion constraint. For example, the disc should not allow excessive loads to be shifted into the facet joints, thereby damaging the balance between the facet joints and the intervertebral disc in the sharing of the load.
3. It should be insertable in such a way as to avoid the destruction of such anatomical structures as the facets and ligaments.
4. It should have excellent endurance as the average patient receiving a lumbar disc replacement is approximately 35 years old, and the prosthesis must last at least 50 years.
5. It should be revisable. An individual will take two million strides per year and perform approximately 125,000 bends. Therefore, in the 50-year life expectancy of a prosthetic disc, there will be greater than 106 million cycles. This is about six-times greater than the expected life cycle of any prosthetic device invented for human use. Accordingly, the device should be easily revised and/or replaced with a new prosthesis with minimal risks to the patient.

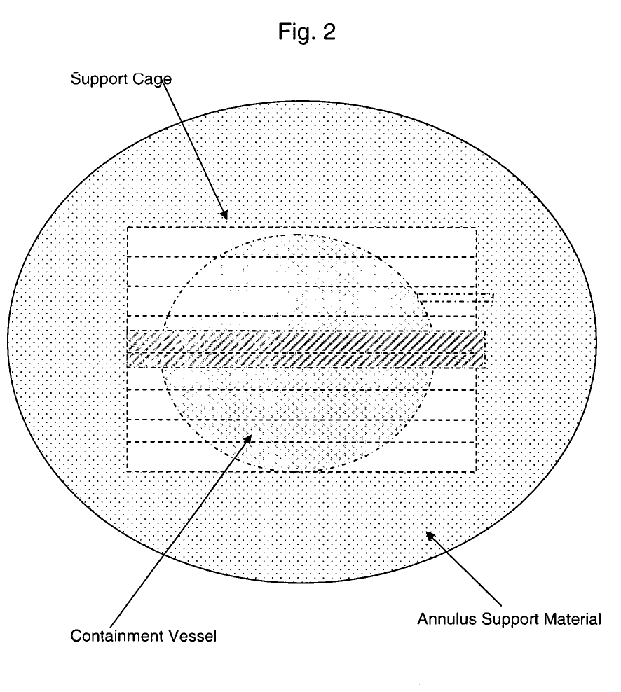

The general concept of the disc prosthesis of the current invention, as shown schematically in FIG. 2, has three main components:
1. A bi-convex support cage, capable of expansion under variable torque control to restore intervertebral body height;
2. A containment vessel within the body of the cage for reinforcing the cage to support axial loads on the order of 1,200 to 1,500 lbs, the containment vessel being designed to be backfilled with orthobiological materials, such as glass polymers, etc., capable of withstanding these axial loads and prevent increased stresses on the posterior facet joint complex; and
3. A suitable annulus support material, such as a hydrogel-type material, which is inserted around the central core of the prosthesis of the current invention to fill the remaining available space within the nucleus such that the inner fibers of the annulus are fully supported and buckling and delamination of the annulus are prevented.

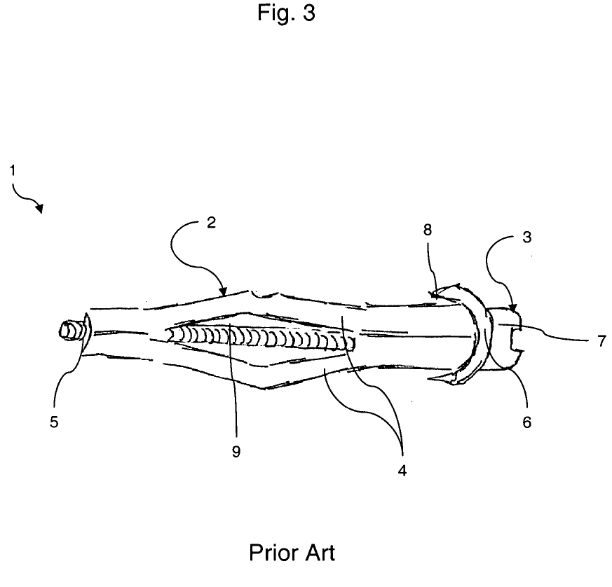

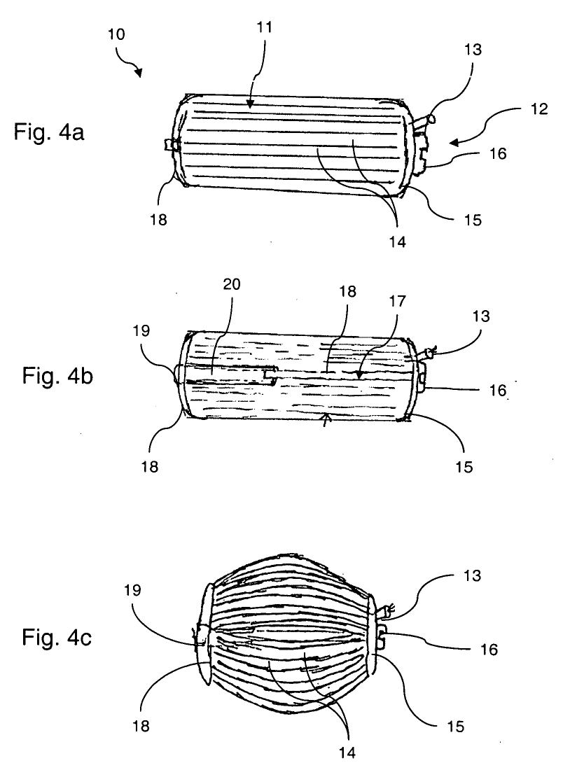

Turning now to the various elements of the prosthesis of the current invention. First, the support cage is roughly related to a Molly-type expansion bolt. Figures comparing the structure and operation of a prior art Molly expansion bolt to the nuclear disc prosthesis of the current invention are provided by FIGS. 3 and 4.

As a comparison, a schematic of a conventional (Molly-type) expansion bolt is provided in FIGS. 3. FIG. 3 shows the expansion bolt (1) in an unexpanded state. As shown, the expansion bolt (1) generally comprises an anchor cage (2) and a screw (3). In this device the cage (2) is made of individual staves (4), which are attached at either end to distal (5) and proximal (6) end caps. The proximal end cap (6) of the anchor cage (2) abuts the head (7) of the screw (3) and is prevented from rotation during expansion by anchor spikes (8), which are driven into a wall. The distal end cap (5) of the anchor cage (2) is threadedly mounted to the threaded shaft (9) of the screw (3) such that as the screw is turned, the distal end cap (5) is pulled along the threaded shaft (9) of the screw toward the head (7) of the screw thereby deforming and expanding the individual staves (4) of the cage outwardly to abut the back end of the wall. Although such a device provides excellent axial pull-out strength, the deformed staves do not provide a significant lateral load bearing structure.

In contrast, as shown in FIGS. 4a to 4c, the disc prosthesis of the current invention is designed around a modified expansion bolt such that more lateral load bearing weight can be provided. FIG. 4a shows a first unexpanded schematic view of the disc prosthesis of the current invention. In this embodiment, the disc prosthesis (10) generally comprises an outer supportive cage (11), and expansion mechanism (12), and a prosthesis fill port (13). Although at initial glance, this device looks on the surface to be very similar to a conventional expansion bolt, the differences can be better understood by examination of the internal portion of the device, which is provided in FIG. 4b. As shown, the supportive cage (11) is formed of a plurality of individual supportive staves (14), which are attached at one end to a proximal end cap (15) which abuts the head (16) of a screw (17), and which allows the threaded shaft (18) thereof to extend in a distal direction through the center of the cage (11). The supportive staves (14) are attached at a second end to a distal end cap (18), which adjoins the end (19) of a threaded sleeve (20) having an elongated sleeve shaft (21) which extends in a proximal direction through the center of the cage (11) to threadedly connect with the threaded shaft (18) of the screw (17). The proximal end cap (15) is designed to allow the free rotation of the screw (17), while the distal endcap is directly coupled to the threaded sleeve (20) to prevent rotation of the threaded sleeve in relation to the staves. Accordingly, during operation (FIG. 4c) as the screw (17) is turned, it is threaded into the sleeve (20), which pulls the distal cap (18) toward the proximal cap (15) along the threaded shaft, thereby outwardly distorting the staves (14) such that an expanded supportive cage is formed.

There are several advantages to the cage design of the current invention. First, by placing an end cap within the threaded sleeve (20) the extent of the distortion of the supportive cage (11) can be controlled such that a specific prosthesis expansion can be achieved. Second, by threading the screw into the sleeve no sharp points, such as the end of the screw in FIG. 1b, are allowed to project outside the disc prosthesis where they might cause damage to nearby tissue. Third, unlike the conventional expansion bolt, which is not reversible, by turning the screw (17) of the disc prosthesis of the current invention in the opposite direction, it can be extracted from the threaded sleeve (20) forcing a straightening of the staves (14) and a collapse of the cage (11). This reversibility allows for the easy removal of a disc prosthesis should it prove defective, or should a more extensive surgical procedure (such as a partial or total discectomy) be required.

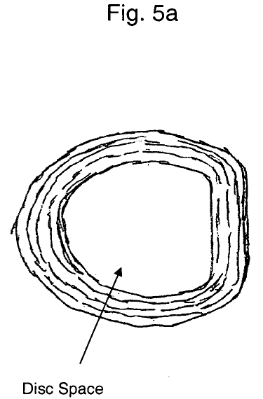

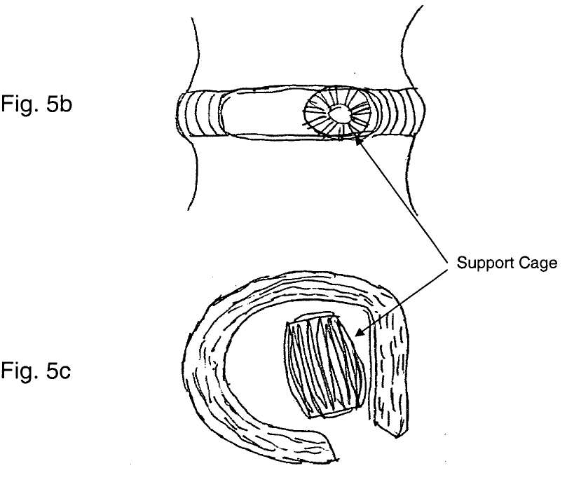

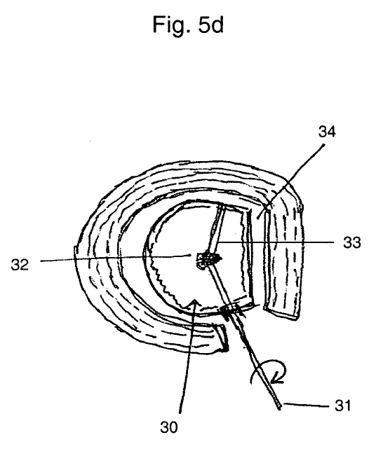

Although the expanded staves, as shown schematically in FIG. 4b above are shown to deform evenly, thus forming a cage having a barrel or distorted cylinder shape, it should be understood that the any suitable cage shape may be used. In one preferred embodiment, the actual cage shape formed by the distorted staves is related to the shape and nature of the disc space to be supported. For example, as shown in FIGS. 5a to 5d, in one embodiment the disc space to be supported is an irregular shape, such as the D-shaped space of a lumbar disc space. In such an embodiment, as shown in FIGS. 5b and 5c, a geometrically regular cylinder would not be of optimal help in supporting the disc space. Accordingly, in one embodiment of the current invention, shown schematically in FIG. 5d, the disc prosthesis would comprise a cage (30) having an angled expansion device (31), whereby interlocked gears (32) or a flexible rod would be used to drive an off-angle expansion rod (33), which in turn would expand the individual support staves (34) of the cage (30). In such an embodiment, the stave would be constructed of an expanded memory metal, such as a titanium-nickel alloy, that when collapsed would be pulled tight against the central expansion drive (31), but upon expansion would “spring” back into its normal and arbitrary memory shape. Using a memory metal allows for the creation of elaborately shaped prosthesis bodies, such as, for example, the D-shaped prosthesis shown schematically in FIG. 5d, above. Such “exotic” shapes allows for a prosthesis with a conformation more closely related to the actual disc space of the body. Such a close conformation facilitates more flexion and extension movements, and also provides for a slight torsional resistance.

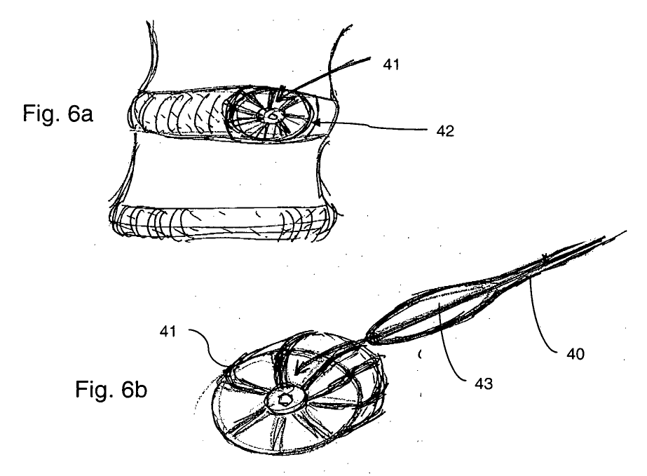

As previously discussed, although the support cage provides some lateral support thereby returning the disc space to a nominal level, the forces produced during normal motion can produce weights on the disc prosthesis of 1,800 to 2,000 lbs, which are far beyond the weight tolerances of the metal supports of the cage. Accordingly, an additional supportive material is needed to provide adequate lateral support for the disc prosthesis. In the current invention this additional support is provided by a collapsible containment vessel disposed in the center of the cage. As shown in FIGS. 6a and 6b, in one embodiment the containment vessel (40) would be inserted in a collapsed fashion within the cage (41) of the disc prosthesis (42). The vessel (40) in turn would be placed into fluid communication with the fill port on the disc prosthesis. The vessel would could then be filled with any suitable material, such as a polymer or a crystalline material, capable of providing sufficient structural support to the disc prosthesis. In one embodiment the vessel would be filled with a high strength polymer material, such as a glass polymer sold under the trade name Cortos, which is capable of achieving the same tensile strength as cortical bone. In another embodiment, a bioactive material, such as a synthetic cancellous bone-void filler material could be used. One such suitable material is an ultra-porous formulation of beta-tricalcium phosphate (B/TCP) sold under the trade name Vitoss. Such materials have been engineered using nanoparticle technology to resemble the structure of cancellous bone. These materials are particularly advantageous as their interconnected micro-porosity facilitates in the in-migration of bone-forming cells and nutrients via enhanced capillary activity. Such materials also are easily manipulated into desirable shapes during surgery. For example, the B/TCP material can be sculpted as a block, or poured as a morsel. In addition, these materials have compressive strengths as high as cortical bone, and therefore, closely match the stiff modulus of the adjacent bony vertebral bodies. Finally, one of the failures of conventional disc prostheses is that they are generally made with high density materials, such as metal, and therefore have a tendency to penetrate the vertebral endplates when placed under pressure. By integrating a filler material that has a density close to that of the adjacent bone, the load will be shared between the prosthesis and the vertebral endplates preventing the type of penetration generally associated with such devices. Although the vessel (40) shown in FIG. 6b is provided in a oblong shape with reinforcing members running along its side (43) to ensure the vessel is able to maintain its shape when filled, it should be understood that any suitable vessel shape may be used in with the disc prosthesis of the current invention.

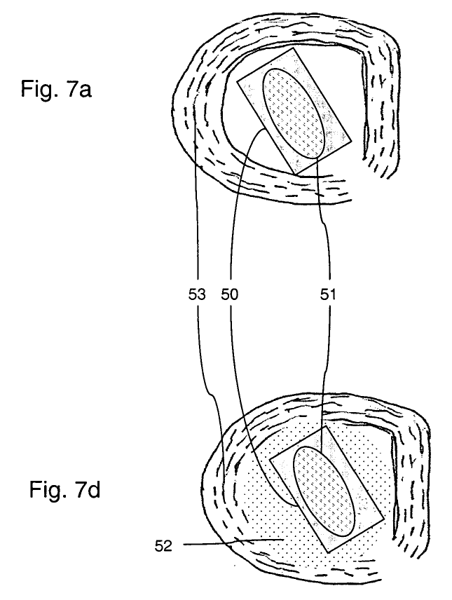

Finally, the prosthesis of the current invention is also designed to mimic the physiological properties of a normal disc. For example, the nucleus of a normal disc has hydrodynamic properties, i.e., the disc is able to imbibe fluid to produce an internal turgor. When a uniaxial load is applied on the disc, the nucleus distributed and converts the uniaxial load into a tangential annular force, which is then attenuated by the viscoelastic properties of the annulus. This phenomenon is also known as “disc creep.” In short, the nucleus acts as an incompressible medium. When the nucleus is excited by mechanical loads of short duration (i.e., less than a second) the nucleus and annulus interact to redistribute and equilibrate the load. As a result, a compressive axial load is seen to cause annular bulging in a momentary manner. Disc creep is the mechanism whereby the disc goes through a process of redistribution until it adapts or reaches a state that is stationary with the experienced loads. Accordingly, to mimic this “internal turgor,” in addition to the cage (50) and the support material (51), a second filler material (52) is backfilled in between the prosthesis cage (50) and the annulus (53), to buttress and support the annulus internally, as shown schematically in FIGS. 7a and 7b. Any material capable of providing the hydrodynamic properties of the nucleus may be used as the filler material, such as, for example, synthetic polymers such as polyvinyl, pyrrolidone, polyvinyl alcohol, poly-2-hydroxyethylmethacrolate, or a polysiloxane modified styrene-ethylene-butylene block copolymer. One such suitable material that is currently used in neurosurgery as a cerebral spinal fluid sealant is sold under the trade name Bio-glue by Cryo-life, Inc. Bio-glue is a combination of bovine albumin and gluteraldehyde that produces large bonding properties. Such a material sets in seconds and can be injected into the disc space to back-fill the area around the disc prosthesis with a conventional glue gun.

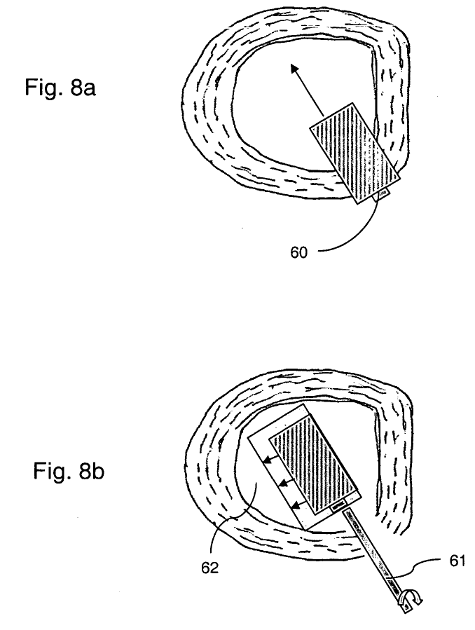

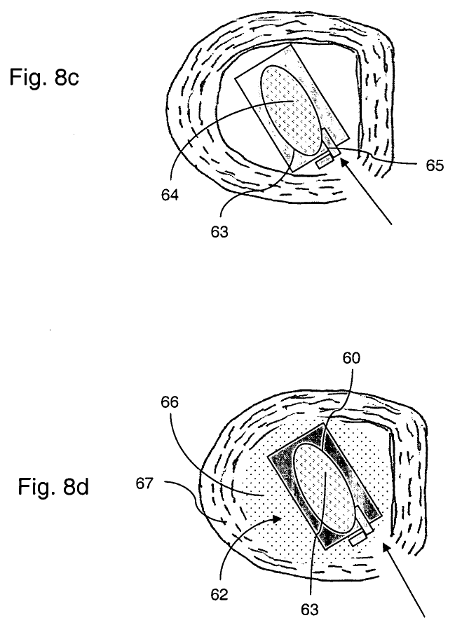

During operation, as shown in FIGS. 8a to 8d, the cage (60) would be inserted into the disc space (FIG. 8a) through a posterolateral approach in the area of the base of the pedicle where the annulus is thicker and more durable. The supportive cage would then be expanded (FIG. 8b) using a variable torque force driver (61) to restore the height of the disc space (62) to a nominal level. Once the supportive cage is expanded to the appropriate height, the containment vessel (63) is then filled with a supportive material (64) through the fill port (65) to ensure adequate structural support of the restored disc space (FIG. 8c). Finally, a filler material (66) is back-filled into the disc space between the disc prosthesis cage (60) and the annulus (67) such that the disc space (62) is finally supported by the combination of the metal supportive cage (60), which is itself supported by the contained structural material (63), which are both surrounded by the hydrodynamic filler material (66), as shown in FIG. 8d.

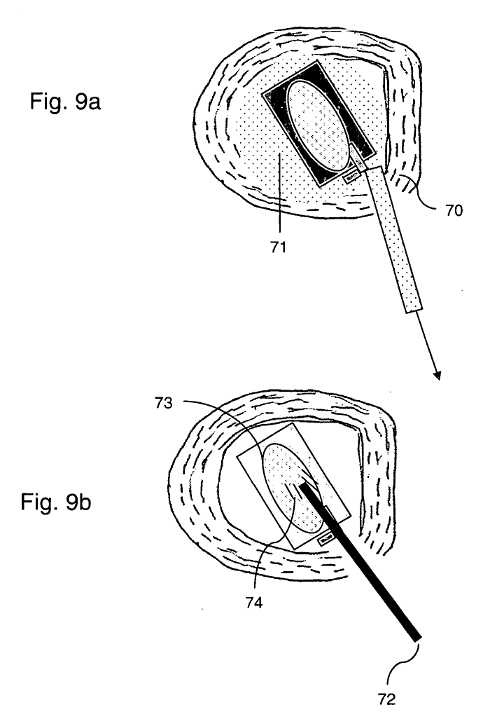

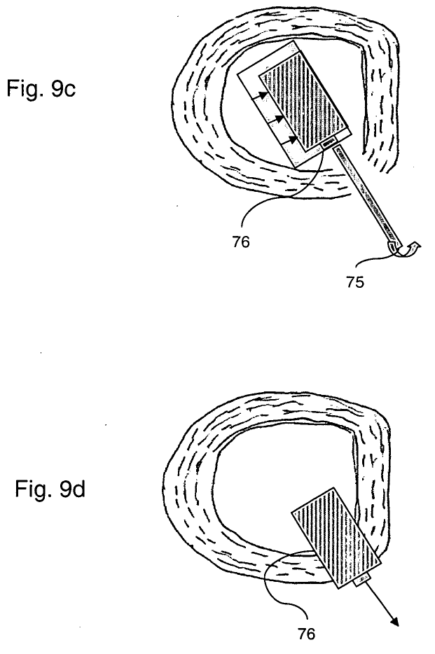

Although the above discussion has only focused on the insertion of the disc prosthesis of the current invention, it is also recognized that given the high stress and strain placed on the prosthesis and the number of use cycles required over the average life of the prosthesis it is likely that the device will ultimately fail. Accordingly, a revision strategy is incorporated into the design of the disc prosthesis of the current invention. The revision of the current disc prosthesis is contemplated in four basic steps as shown schematically in FIGS. 9a to 9d. First, as shown in FIG. 9a, an incision is made in the annulus (70) and the filler material (71) is sucked out of the disc space using any suitable surgical suction device. Once the filler material is removed, as shown in FIG. 9b, an ultrasonic probe or wand (72) is inserted inside the containment vessel (73) to break down the structural material (74) contained therein so that the contents of the containment vessel can be removed from within the disc space. The use of ultrasonic vibration to destabilize materials and allow their extraction has been used in opthamalogical practice to emulsify a defective lens in cataract surgery. In one such embodiment, the ultrasonic probe would be vibrated at 18,000 to 62,000 cycles/per/second to emulsify the structural support material, allowing it to be washed and suctioned out of the containment vessel. Once the containment vessel (73) has been emptied, as shown in FIG. 9c, a torque tool (75) may be inserted into the disc space to contract the prosthesis cage (76) so that is can be removed from the disc space completely. Once the disc prosthesis is fully removed any suitable replacement prosthesis can be inserted into the evacuated disc space as shown in FIG. 9d. Although a secondary nuclear disc prosthesis of the current invention could be inserted, it is also possible that the continued wear on the disc space and surrounding bones will require the insertion of a intervertebral disc prosthesis for interbody fusion device, or to allow for a total disc replacement, which entails the removal of the vertebral endplates and annulus, and posterior and anterior longitudinal ligaments to completely replace the intervertebral disc space.

Although the above “revision” discussion has focused on the complete removal of the nuclear disc prosthesis of the current invention prior to implanting a revised prosthesis device, in an alternative embodiment, portions of the nuclear disc prosthesis could be retained for use as the basis of an interbody fusion. In this embodiment, the steps shown in FIGS. 9a and 9b remain unchanged. Namely, first the filler material is removed followed by the ultrasonic break-up and removal of the structural material. However, rather than removing the cage of the disc prosthesis, in this more minimal revision a fusion material, such as a bone morphogenic protein or other biologically active substance is pumped into the disc space to fuse the two adjoining vertebral bodies together. Such a revision would be of particular economy in those cases where the staves of the prosthesis cage are formed of nickel and titanium, as these materials are very compatible with bone and may over time themselves adhere to the adjoining vertebral bodies.

Although specific embodiments are disclosed herein, it is expected that persons skilled in the art can and will design alternative nuclear disc prostheses and methods that are within the scope of the following claims either literally or under the Doctrine of Equivalents.

What is claimed is:

1. A disc nucleus prosthesis comprising:
a support cage comprising a proximal cap, a distal cap, and a plurality of deformable staves coupled between the proximal and distal caps, wherein the distal cap includes a threaded sleeve that extends in a proximal direction along the center axis of the support cage; and
a threaded shaft extending through the proximal cap in a distal direction along the center axis of the support cage, the threads of the threaded shaft being threadedly coupled to the threaded sleeve such that when the threaded shaft is rotated, the proximal and distal caps are moved closer or farther apart causing a deformation in the plurality of staves.

2. The disc nucleus prosthesis of claim 1, wherein the threaded shaft further includes a head proximal to the proximal cap, wherein the head is designed to provide a coupling point for a torquing tool.

3. The disc nucleus prosthesis of claim 1, wherein the distal cap is the threaded aperture and is formed from a portion of the threaded sleeve extending toward the proximal cap.

4. The disc nucleus prosthesis of claim 1, wherein the plurality of staves are flexible and curve outward from the center of the housing when the proximal cap and the distal cap are moved toward each other, and wherein the deformable legs straighten when the proximal cap and the distal cap are moved away from each other.

5. The disc nucleus prosthesis of claim 1, wherein the plurality of staves are made of a memory alloy, such that each of the staves are released into a pre-set memory position of arbitrary geometric shape when the proximal cap and the distal cap are moved toward each other, and wherein the staves are straighten out of said pre-set memory position when the proximal cap and the distal cap are moved away from each other.

6. The disc nucleus prosthesis of claim 5, wherein the memory alloy is a nickel-titanium alloy.

7. The disc nucleus prosthesis of claim 1, further comprising:
an expandable containment vessel disposed within the support cage; and
a fill port in fluid communication through the support cage to the containment vessel.

8. The disc nucleus prosthesis of claim 7, further comprising a support material capable of withstanding an axial load of from 1,200 to 1,500 lbs disposed within the containment vessel, the fill port further comprising a valve to prevent the exit of the support material from the containment vessel.

9. The disc nucleus prosthesis of claim 8, wherein the support material is selected from the group consisting of an orthobiological material or a glass polymer.

10. The disc nucleus prosthesis of claim 9, wherein the orthobiological material is a synthetic cancellous bone-void filler material.

11. The disc nucleus prosthesis of claim 10, wherein the synthetic cancellous bone-void filler material is beta-tricalcium phosphate.

12. The disc nucleus prosthesis of claim 1, further comprising a hydrodynamic filler material disposed between the support cage and the surrounding anatomical structures.

13. The disc nucleus prosthesis of claim 12, wherein the hydrodynamic filler material is selected from the group consisting of synthetic polymers, sealants and tissue adhesives.

14. The disc nucleus prosthesis of claim 12, wherein the hydrodynamic filler material is a combination of bovine albumin and gluteraldehyde.

15. A disc nucleus prosthesis comprising:
a support cage comprising a proximal cap, a distal cap, and a plurality of deformable staves coupled between the proximal and distal caps, wherein the distal cap includes a threaded sleeve that extends in a proximal direction along the center axis of the support cage;
a threaded shaft extending through the proximal cap in a distal direction along the center axis of the support cage, the threads of the threaded shaft being threadedly coupled to the threaded sleeve such that when the threaded shaft is rotated, the proximal and distal caps are moved closer or farther apart causing a deformation in the plurality of staves;
an expandable containment vessel disposed within the support cage;
a fill port in fluid communication through the support cage to the containment vessel, the fill port further comprising a valve to seal the containment vessel;
a support material capable of withstanding an axial load of from 1,200 to 1,500 lbs disposed within the containment vessel; and
a hydrodynamic filler material disposed between the support cage and the surrounding anatomical structures.

16. A method for posterolateral insertion of the disc prosthesis of claim 15 into a disc space comprising the steps of:
making an incision at the base of the pedicle to form an opening in the annulus of the nucleus of a disc;
inserting the support cage through said incision into said disc space;
expanding said cage by application of a torque to said threaded shaft such that the nominal vertical height of the disc space is restored;
inserting of said support material into said containment vessel through said fill port;
backfilling said remaining disc space with said hydrodynamic filler material; and
closing said incision.

17. A method of revising the disc prosthesis of claim 15 comprising the steps of:
making an incision at the base of the pedicle to form an opening in the annulus of the nucleus of a disc;
suctioning out the hydrodynamic filler surrounding the support cage;
inserting an ultrasonic probe into said containment vessel, and activating said probe to emulsify the support material; and
removing said emulsified support material from said containment vessel.

18. The method of claim 17, further comprising the step of:
filling the remaining disc space with a biological active bone fusion material to fuse the adjoining intervertebral bodies.

19. The method of claim 18, wherein the bone fusion material is a bone morphogenic protein.

20. The method of claim 17, further comprising the steps of:
collapsing said cage by application of a torque to said threaded shaft such that the cage is returned to its unexpanded shape; and
removing said collapsed cage through said incision.
